(19) United States
(12) Patent Application Publication
(10) Pub. No.: US 2018/0070554 A1
(43) Pub. Date: Mar. 15, 2018

(54) BARN CEILING GRID SYSTEM

(71) Applicants: Jerry Kloostra, Belmont, MI (US); Janet Kloostra, Belmont, MI (US)

(72) Inventors: Jerry Kloostra, Belmont, MI (US); Janet Kloostra, Belmont, MI (US)

(21) Appl. No.: 15/263,745

(22) Filed: Sep. 13, 2016

(51) Int. Cl. A01K 1/00; A01K 29/00; H01F 7/20; A61N 2/02; A61D 99/00

(52) U.S. Cl. CPC A01K 1/00; A01K 29/00; A61D 99/00; A61N 2/02; H01F 7/202

(57) ABSTRACT

A barn ceiling grid system for providing a magnetic environment in a barn for enhancing biological process in the living organism is disclosed. The barn ceiling grid system comprises two rods, a wire, and a power source. Each rod is positioned along opposing edges of a barn ceiling, and the wire is interconnected between the rods in a predetermined pattern to generate a network of wiring across the rods. The power source supplies an electrical current, which is passed through the predetermined pattern of the wire to generate an electromagnetic field. The electromagnetic field is induced under a space defined below the network of the wiring across the length of the rods. The generated electromagnetic field provides a magnetic environment for the living organisms underneath the system. The system creating a magnetic environment provides a euphoric contentment for the living organisms, and improves their growth, milk production, egg production, and reproduction process.

### BACKGROUND OF THE INVENTION

### A. Technical Field

The present invention generally relates to a system configured for a barn ceiling grid, and more specifically relates to the system for providing a magnetic environment configured in the barn ceiling grid for any living organisms, which includes and not limited to, dairy cattle, beef cattle, chicken and egg production, turkeys, all domestic animals, including Zoo animals and breeding facilities, including horse training facilities this environment will creates a calming, euphoric effect, and enhances the growth and biological process in the living organisms.

### B. Description of Related Art

In the past, farming and animal husbandry made efforts to optimize milk production, and growth of the living organisms through genetics and feed manipulation. Currently, the use of an electromagnetic energy is attempted to enhance the growth and biological process in the living organisms. However, many of the techniques have been limited to belts, pads or mats, which apply magnetic or electromagnetic energy to the living organisms.

The disadvantages of these techniques include the necessity for the organism to wear the wearable devices with a portable power source in order to generate the electromagnetic energy to the living organisms. Some of the proposed prior arts attempted to affect the environment surrounding the organism are, Cuppen, U.S. patent application Ser. No. 12/670,728 discloses a distributed coil structure for applying a time varying electromagnetic field to a population of living organism. Miller et. al., U.S. Pat. No. 6,203,486 discloses a magnetic-field augmenter hung on a wall, floor, or around seating furniture in the buildings with additional protective device against loose connections and shorts in the power source. Wilhelm et. al., U.S. patent application Ser. No. 10/701,395 describes a magnetic field application device attached on a region of the housing for providing magnetic field to provide a curative effect on the humans or animals inside the housing.

Baugh, U.S. Pat. No. 6,902,521 discloses a system and method of enhancing growth and biological functions of the living systems through an electric wire coupled to the electromagnetic generator with an air pump and hose to aerate a body of water within an environment for increasing an oxygen content of said body of water. However, the known arts requires additional expensive equipment for protection and aeration, and the electromagnetic generator is fitted to the buildings consume additional maintenance cost for the users.

Therefore, there is a need in the art for a system to create a complete enhanced magnetic environment for the living organisms with simple and inexpensive construction. When a dairy cow, beef steer, horse or any creature living or using this magnetic environment facility their water, body fluids and particularly the blood cells become organized and can more effectively deliver more energy and oxygen. The other effect this environment has on these living creatures is a calming effect, like a euphoric contentment. These two effects will greatly enhance, milk production, beef cattle weight gain, horse training for its calming effect, Zoo reproductive facilities, egg production, and raising chickens and turkeys. Most large cattle farms have 1,000 cattle per barn.

### SUMMARY OF THE INVENTION

The present invention relates to a barn ceiling grid system, which is implemented in an inner space of a barn. In an embodiment, the barn ceiling grid system comprises at least two rods, a wire, and a power source. In one embodiment, each rod is positioned along opposing edges of a barn ceiling. In one embodiment, the wire is interconnected between the two rods in a predetermined pattern to generate a network of wiring across the length of the two rods. In one embodiment, the power source supplies an electrical current, which is passed through the predetermined pattern of the wire to generate an electromagnetic field. In one embodiment, the electromagnetic field is induced under a space defined below the network of the wiring across the length of the two rods. In one embodiment, the generated electromagnetic field provides a magnetic environment for one or more living organisms underneath the system.

In an embodiment, the rods are segmented into one or more sections positioned along the opposing edges of the barn ceiling. In another embodiment, the rods are segmented in the range from 2 to 10 sections positioned along the opposing edges of the barn ceiling. In one embodiment, the size of the rods are in predetermined size positioned along the opposing edges of the barn ceiling. In an embodiment, the wire interconnected between the two rods are arranged in a zig-zag pattern to generate the network of wiring across the length of the two rods. In another embodiment, the wire interconnected between the two rods are arranged in a coiled pattern to generate the network of wiring across the length of the two rods. In an embodiment, the electromagnetic field is induced is in the range of 0.5 to 50 Gauss induced under a space defined below the network of wiring across the length of the two rods.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 shows a perspective view of the barn ceiling grid system implemented in the ceiling of the barn according to an embodiment.

FIG. 2 shows a perspective view of the barn ceiling grid system with a zig-zag pattern of wires interconnecting the rods according to an alternate embodiment.

FIG. 3 shows a top view of the barn ceiling grid system with a segmented rod according to an alternate embodiment.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore indicated by the appended claims rather than by the foregoing description. All changes that come within the meaning and range of equivalency of the claims are to be embraced within their scope.

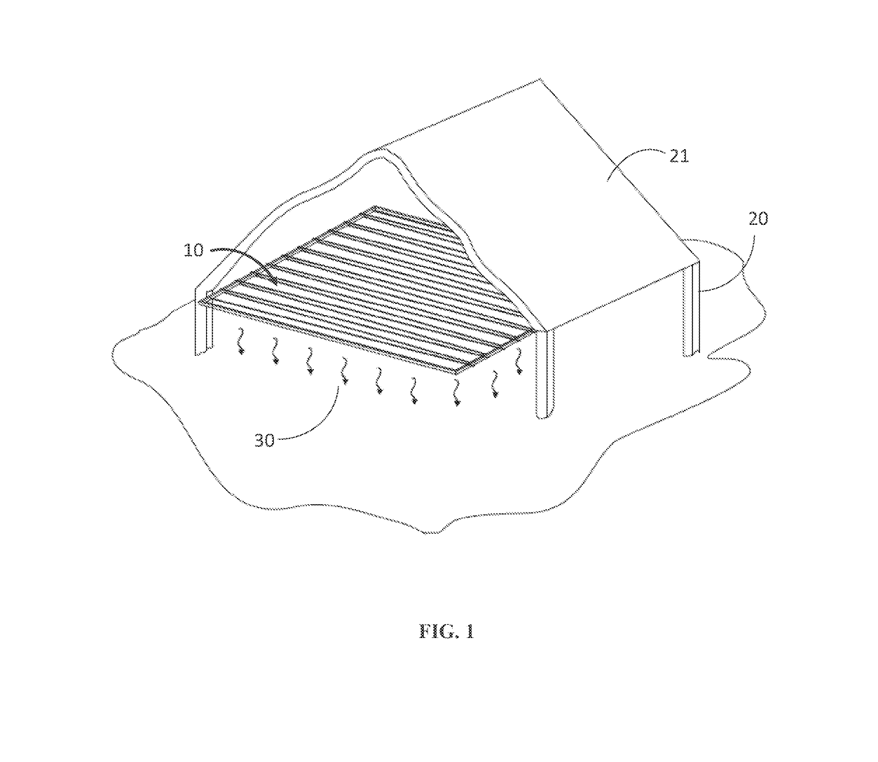

The present invention is directed to a barn ceiling grid system 10, which is implemented in an inner space of a barn 20. Referring to FIG. 1, showing a perspective view of the barn ceiling grid system 10 providing a magnetic field 30 implemented in the ceiling 21 of the inner space of the barn 20 or shelter for any living organisms such as hens, cows, sheep or even human beings.

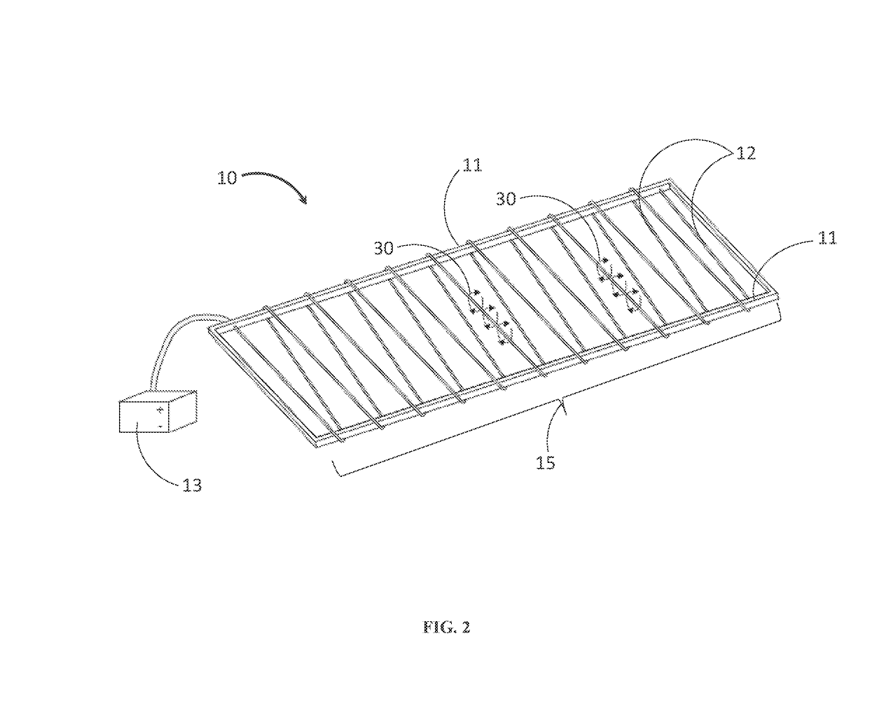

Referring to FIG. 2, showing a perspective view of the barn ceiling grid system 10 implemented in the ceiling 21 of the inner space of the barn 20. In an embodiment, the barn ceiling grid system 10 comprises at least two rods 11, a wire 12, and a power source 13. In an embodiment, each rod 11 is positioned along the opposing edges of the barn ceiling 21 in the proposed system 10. In one embodiment, the wire 12 is interconnected between the two rods 11 in a predetermined pattern to generate a network 15 of wiring across the length of the two rods 11. In one embodiment, the power source 13 supplies an electrical current, which is passed through the predetermined pattern of the wire 12 to generate an electromagnetic field 30. In one embodiment, the electromagnetic field 30 is induced under a space defined below the network 15 of the wiring across the length of the two rods 11. In one embodiment, the generated electromagnetic field 30 provides a magnetic environment for one or more living organisms underneath the system 10. In one embodiment, the power source 13 is an uninterrupted power source from any kind of known existing energy.

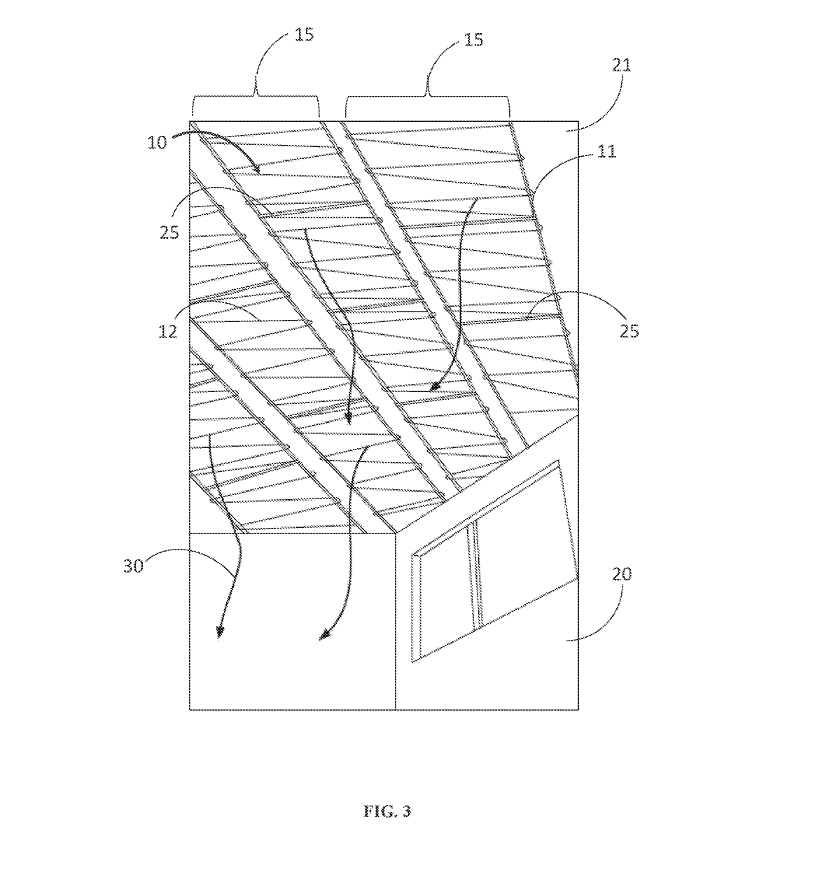

Referring to FIG. 3, showing a perspective view of the barn ceiling grid system 10 with a segmented rod 11 according to an embodiment of the proposed invention. In an embodiment, the rods 11 are segmented into one or more sections 25 positioned along the opposing edges of the barn ceiling 21. In another embodiment, the rods 11 are segmented in the range from 2 to 10 sections 25 positioned along the opposing edges of the barn ceiling 21. In one embodiment, the size of the rods 11 are in predetermined size positioned along the opposing edges of the barn ceiling 21. In one embodiment, the rods 11 are segmented in horizontal, vertical or crosswise sections configured to position positioned along the opposing edges of the barn ceiling 21.

In an embodiment, the wire 12 interconnected between the two rods 11 are arranged in a zig-zag pattern to generate the network 15 of wiring across the length of the two rods 11. In another embodiment, the wire 12 interconnected between the two rods 11 are arranged in a coiled pattern to generate the network 15 of wiring across the length of the two rods 11. In one embodiment, the pattern of wires 12 interconnecting the rods 11 is customized with any patterns for an aesthetic look in the barn ceiling 21.

In an embodiment, the electromagnetic field 30 or magnetic field is induced is in the range of 0.5 to 50 Gauss induced under a space defined below the network 15 of wiring across the length of the two rods 11. In one embodiment, the magnetic field 30 is similar in Gauss strength used in the existing known arts such as human magnetic products, mattresses, wraps or bracelets. In another embodiment, the strength of the magnetic field 30 is maintained to provide the barn 20 or environment surrounding the living organisms with magnetic energy.

The advantage of the barn ceiling grid system 10 is simple in construction, and cost-effective comparing to the existing arts. The barn ceiling grid system 10 provides a centralized and uninterrupted enhanced magnetic environment for the living organisms. The system 10 creating a magnetic environment dramatically organizes the flow of blood in the cells and efficiently carries more energy and oxygen, which provides a calm feel with euphoric contentment for the living organisms. The system 10 further helps in enhancing the milk production, beef cattle raising farms, horse training facilities, egg production, chicken and turkey farms and reproduction of the exotic organisms in the zoo environment.

The foregoing description comprises an illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A barn ceiling grid system implemented in an inner space of a barn, wherein barn ceiling grid system comprises;
at least two rods, wherein each rod is positioned along opposing edges of a barn ceiling;
a wire interconnected between the two rods in a predetermined pattern to generate a network of wiring across the length of the two rods; and
a power source, wherein the power source supplying an electrical current passed through the predetermined pattern of wire, wherein an electromagnetic field is induced under a space defined below the network, wherein the generated electromagnetic field provides a magnetic environment for one or more living organisms underneath the system.

2. The barn ceiling grid system of claim A1, wherein the rods are segmented in one or more sections positioned along the opposing edges of the barn ceiling.

3. The barn ceiling grid system of claim A1, wherein the rods are segmented in the range from 2 to 10 sections positioned along the opposing edges of the barn ceiling.

4. The barn ceiling grid system of claim A1, wherein the rods are in predetermined size positioned along the opposing edges of the barn ceiling.

5. The barn ceiling grid system of claim A1, wherein the wire interconnected between the two rods are arranged in a zig-zag pattern to generate the network of wiring across the length of the two rods.

6. The barn ceiling grid system of claim A1, wherein the wire interconnected between the two rods are arranged in a coiled pattern to generate the network of wiring across the length of the two rods.

7. The barn ceiling grid system of claim A1, wherein the electromagnetic field is induced is in the range of 0.5 to 50 Gauss induced under a space defined below the network of wiring across the length of the two rods.
